(19) United States
(12) Patent Application Publication
(10) Pub. No.: US 2004/0053341 A1
(43) Pub. Date: Mar. 18, 2004

(54) Method for treating histological and biological samples

(76) Inventor: Ilia Borisovitch Izvoztchikov, St. Petersburg (RU)

Correspondence Address: Kohler Schmid + Partner, Ruppmannstr 27, Stuttgart, D 70565, DE

(21) Appl. No.: 10/470,304

(22) PCT Filed: Feb. 11, 2002
(86) PCT No.: PCT/RU02/00056

(30) Foreign Application Priority Data
Feb. 14, 2001 (RU) 2001105043

(51) Int. Cl. G01N 33/53; G01N 33/567; G01N 1/30; G01N 33/48

(52) U.S. Cl. 435/7.2; 435/40.5

(57) ABSTRACT

A method of processing histological and biological specimens includes the sequential immersion of specimens into an aqueous solution of fixing agent, into at least three dehydrating liquids containing water and into at least three waterless dehydrating liquids. A chemical marker is added to the water contained in the aqueous solution of fixing agent, and a chemical indicator, forming a colored composition with the chemical marker, is added to the waterless dehydrating liquid. On appearance of a visually perceptible coloring in the waterless dehydrating liquid, that liquid is renewed. Ethyl and isopropyl alcohols or mixtures based thereon are used as dehydrating liquids. Transition metal salts having an oxidation rate of 2+ are used as chemical markers, such as salts of iron, cobalt, nickel, copper and zinc in the amount of 2-40 g (on the basis of metal) per liter of water. 1,10-phenanthroline, diphenylthiocarbazone, 1-nitroso-2-naphthol, sodium diethyldithiocarbamate, 8-oxyquinoline, 1-(2-pyridilazo)-2-naphthol, 4-(2-pyridilazo)-resorcinol or 2-carboxy-2′-hydroxy-5′-sulfoformazylbenzol are used as indicators in the amount of 0.001-0.4 g per liter of alcohol, or potassium or sodium thiocyanate in the amount of 5-10 g per liter of alcohol.

True Translation of PCT/RU02/00056 as filed on Feb. 11, 2002

### TECHNICAL FIELD

The invention concerns a method of diagnosis, in particular a method for preparation of histological and biological specimens prior to microscopic examination.

Common preparation of tissues for microscopic examination includes the paraffin impregnation of tissue specimens, obtaining a microscopic section of the impregnated specimen, mounting this section on the object slide, de-waxing, section staining and its introduction into the optical path. The specimen having been prepared in such a way is examined under the microscope. Specimen impregnation is required in order to prevent tissue crumpling while obtaining the section and to retain the tissue and cellular structures being examined. However, tissue paraffin impregnation requires dehydration, since tissues containing water are either poorly impregnated with paraffin or not impregnated at all. Tissue dehydration is performed in a liquid mixable with water, and this liquid then has to be replaced with a paraffin solvent mixable with dehydrating liquid.

### PRIOR ART

A method of histological specimen dehydration is known using dehydration in 1,4-diethylenedioxide (dioxane) [B. Romeys, Microscopic Technique, M. <<FL>>, 1953, pp. 81-82]. Dioxane is well mixable both with water and paraffin so it is not necessary to process it with a second solvent in order to displace the dehydrating solvent before waxing.

Dioxane is, however, inflammable and highly toxic. The dioxane toxicity has a cumulative nature, and a human being becomes rapidly accustomed to the odor of dioxane and is no longer aware of its presence. Since its use resulted in poisoning, dehydration with dioxane has not become widespread.

Another method of dehydration consists of fourfold curing of histological specimens in acetone [R. Lilly. Pato-histological equipment and practical histo-chemistry. M., <<Mir>>, 1969, p. 69]. The dehydration in acetone is performed rapidly: it is sufficient to cure the specimen in each portion of solvent for 20 minutes, provided fresh acetone is used for each stage. However, the use of fresh solvent for each stage is expensive. Consequently, fresh acetone is used in the fourth stage only, the vessels are shifted by one position, and acetone that has been used four times is purified by distillation. During dehydration according to this scheme the specimens are cured for 40 minutes in each stage.

Dehydration with acetone has the disadvantage that the acetone upsets the tissue and cellular structures. Hence, dehydration with acetone is used only occasionally when rapid examination is required.

The most common method of dehydration is the method of histological and biological specimen processing by way of sequential specimen immersion into a water solution of fixing agent, at least into three portions of dehydrating liquid containing water and, at least into three portions of completely waterless dehydrating liquid [R. Lilly. Pato-histological equipment and practical histo-chemistry. M., <<Mir>>, 1969, p. 69]. The most commonly used fixing agent is 4% aqueous solution of formaldehyde. In this method, ethyl and isopropyl alcohols or their mixtures are used as dehydrating liquids. The method permits substitution of the water contained in the tissues and cells of histological and biological specimens without destruction of their tissue and cellular structures. This method is widely used in laboratories for both mechanized and automated processing of histological and biological specimens [e.g., see ER 0 077477 published in 1983; ER No. 0269316 published in 1988; German patent No. 3042578 published in 1987; U.S. Pat. No. 4,576,796 published in 1986; U.S. Pat. No. 4,911,915 published in 1990; RF patent No. 2150097 published in 2000; Bulletin of Inventions No. 15].

During the dehydration process, the water, fats, proteins etc. contained in the tissues enter into the processing liquids. Accumulation of these components in the processing liquids causes a decrease in the processing rate and in the specimen quality. If the contaminated liquids are used for a long time, areas beyond examination can appear in the specimens [see S. W. Thomson, L. G. Luna. An Atlas of Artifacts Encountered in the Preparation of Microscopic Tissue Sections. Ch. C. Thomas Publishers, USA, 1978, pp. 76-77]. In highly active laboratories, renewal of alcohols is usually performed twice a week. As in the case with acetone, the primary (most diluted and contaminated) liquid is poured out, the vessels are shifted by one position and the last one is filled up with fresh waterless liquid.

However, tissues of differing volume must be processed. Endoscopic and punch biopsy specimens have volumes many times less than those of specimens taken during examination of operation or autopsy materials (0.001-0.01 cm³ and up to 1 cm³, respectively). There are no reliable criteria of liquid status estimation and conventional methods often lead to liquid renewal long before its efficiency is terminated. This, in turn, causes unnecessary expense and an increase in the contaminated liquid volume, whose disposal also entails additional costs.

### DISCLOSURE OF INVENTION

Is the underlying object of the instant invention to efficiently monitor and control dehydrating liquid while maintaining high processing quality.

In addition, the object of the invention is also to reduce disadvantageous expense associated with dehydrating liquids, while maintaining high processing quality.

According to the invention, a method of microscopic and biological specimens processing is proposed using sequential immersion of specimens into the processing liquids containing the water solution of the fixing agent, with at least three dehydrating liquids containing water and at least three waterless dehydrating liquids. The method proposes addition of chemical marker into the water contained in the fixing agent water solution, and addition of indicator, forming a colored composition with the chemical marker, into the waterless dehydrating liquid. When coloring of the waterless dehydrating liquid is perceived, the latter should be renewed.

Water and alcohol soluble transition metal salts having an oxidation rate of 2+ are used as the chemical marker in the method: for example, salts of iron (II), cobalt (II), nickel (II), copper (II) or zinc (II).

Zinc has no oxidation rate exceeding 2+, but like cadmium and mercury, it does not belong to the elements of main subgroups of Mendeleev's chart. Formally it belongs to the transition metals [e.g., see Cotton F. A. and Wilkinson G. Basic inorganic chemistry. <<John Wiley and Sons>>, N.-Y., 2.6; 22.1].

Chlorides, sulfates and acetates of these metals are soluble in water and alcohols. The amount of salt added to the fixing agent water solution is 2-40 g per metal ion per 1 liter of water.

Substances like 1,10-phenanthroline, diphenylthiocarbazone, 1-nitroso2-naphthol, sodium diethyidithiocarbamate, 8-oxyquinoline, 1-(2-pyridilazo)-2-naphthol, 4-(2-pyridilazo)-resorcinol and 2-carboxy-2′-hydroxy-5′-sulfoformazylbenzol taken at concentration of 0.001-0.4 g/l are used as indicators. It is also possible to use potassium and sodium thiocyanates as indicators at concentration of 5-10 g/l.

Alcohols such as ethyl or isopropyl, or alcohol mixtures are used as the dehydrating liquid in the present method: for example, ethyl and methyl alcohols or ethyl and methyl alcohols with the addition of acetone. Ethyl and isopropyl alcohols are preferred.

The colored reactions of the metals used are known with the indicators being used in water, chloroform, benzene, cyclohexane and carbon tetrachloride [E. Sendal. Colorimetrical methods of detection of metal traces. <<Mir>>, M., 1964; Y. Y. Lurye. Reference book on analytical chemistry. <<Himiya>>, M., 1979]. However, these reactions have not been examined in alcohols and neither the colors of the compositions formed nor the concentration detection limits were known.

Formaldehyde (4% aqueous solution) or mixtures based thereon are preferably used as a fixing agent.

The inventive method is illustrated by but not limited to the following examples.

### EXAMPLE 1

300 cassettes each containing a single endoscopic specimen with a volume of 0.001 cm³ are loaded into the device for processing microscopic specimens. The total specimen volume is 0.3 cm³. A battery of process liquids includes 7 containers each having a volume of 3 liters containing, respectfully:

| | |
|---|---|
| 1. | Fixing agent: 4% water solution of formaldehyde |
| | containing 39.85 g/l (8 g/l on the basis of iron) |
| | of FeSO₄·7H₂O; |
| 2.) | 70% water solution of ethyl alcohol; |
| 3.) | 80% water solution of ethyl alcohol; |
| 4.) | 90% water solution of ethyl alcohol; |
| 5, 6 and 7.) | 100% ethyl alcohol containing 0.2 g/l of |
| | 1,10-phenanthroline. |

The liquids are fed into the device where the fixing agent remains for 8 hours and the alcohol solutions—for 1 hour.

The amount of liquid transferred from a preceding container into a subsequent one depends on the amount of liquid contained in the specimens and the amount of liquid carried on the surface of cassettes and apparatus parts (walls of container and pipeline). The latter amount is constant for a given apparatus and assumes a value of 25 ml.

After processing the specified specimens, no liquid color occurred in the 5^{th} container.

14 batches of the endoscopic specimens, 300 pieces each, were processed in a similar way. The apparent red-orange coloring of the alcohol in the 5^{th} container occurred after the 15^{th} batch processing.

Microscopic examination of the specimens has shown that the specimens in all of fifteen batches were processed with high quality.

### EXAMPLE 2

The experiment was conducted as in Example 1, but the device was loaded with autopsy specimens having a volume of about 0.7 cm³. The total volume of the tissues being processed was 210 cm³. Coloring occurred in the 5^{th} container after processing of the third batch.

Microscopic examination has shown that the specimens were dehydrated with high quality.

### EXAMPLE 3

The experiment is conducted as in the Example 1, but the dehydration is performed with isopropyl alcohol. 1,10-phenanthroline at 0.4 g/l concentration is introduced into the waterless isopropyl alcohol.

After the fifteenth cycle of fixation and dehydration, the apparent yellow coloring appears in the fifth container. The alcohol is renewed.

Microscopic examination has shown that the specimens were dehydrated with high quality.

### EXAMPLE 4

The experiment is conducted as in Example 3, but 300 autopsy specimens are processed with a total volume of 270 cm³. A noticeable coloring occurs in the 5^{th} container after the third dehydration cycle.

Microscopic examination has shown that the specimens were dehydrated with high quality.

### EXAMPLE 5

The experiment is conducted as in Example 1, but 8.8 g/l (2 g/l on the basis of zinc) of the zinc sulfate (ZnSO₄.7H₂O) is introduced into the fixing agent solution. 0.002 g/l of diphenylthiocarbazone is introduced into the waterless ethyl alcohol.

After the 15^{th} processing cycle, a noticeable magenta-red coloring occurs in the fifth container. The alcohol is renewed.

Microscopic examination has shown that the specimens were dehydrated with high quality.

### EXAMPLE 6

The experiment is conducted as in Example 3, but 70.4 g/l (16 g/l on the basis of zinc) of zinc sulfate is introduced into the fixing agent solution. 0.001 g/l of diphenylthiocarbazone is introduced into the waterless isopropyl alcohol.

After the thirteenth processing cycle, a red coloring is apparent in the fifth container.

### EXAMPLE 7

The experiment is conducted as in Example 2, but 57.25 g/l (12 g/l on the basis of cobalt) of the cobalt sulfate (CoSO₄.7H₂O) is introduced into the fixing agent solution, and 10 g/l of potassium thiocyanate is dissolved in the waterless ethyl alcohol.

After the third processing cycle a blue coloring is apparent in the fifth container.

### EXAMPLE 8

The experiment is conducted as in Example 7, but 0.02 g/l of 1-nitroso-2-naphthol is introduced into the waterless ethyl alcohol.

After the third processing cycle, a reddish-brown (like strong tea) coloring is apparent in the fifth container.

### EXAMPLE 9

The experiment is conducted as in Example 2, but 34.30 g/l (12 g/l on the basis of copper) of copper acetate is introduced into the fixing agent solution. 0.04 g/l of sodium diethyidithiocarbamate is introduced into the waterless ethyl alcohol.

After the third processing cycle, a yellowish-green coloring is apparent in the fifth container.

### EXAMPLE 10

The experiment is conducted as in Example 9, but 114.34 g/l (40 g/l on the basis of copper) of copper acetate is used, and 0.02 g/l of 8-oxyquinoline is added into the waterless ethyl alcohol.

After the third processing cycle, a green coloring is apparent in the fifth container.

### EXAMPLE 11

The experiment is conducted as in Example 2 but 38.27 g/l (8 g/l on the basis of nickel) of nickel sulfate NiSO₄.7H₂O is introduced into the fixing agent solution. 0.004 g/l of diphenylthiocarbazone is introduced into the waterless ethyl alcohol.

After the third processing cycle, a violet coloring is apparent in the fifth container.

### EXAMPLE 12

The experiment is conducted as in Example 4, but 52.8 g/l (12 g/l on the basis of zinc) of zinc sulfate is introduced into the fixing agent solution. 0.01 g/l of 1-(2-pyridilazo)-2-naphthol is introduced into the waterless isopropyl alcohol.

After the third processing cycle, a red-brown (like the strong tea) coloring is apparent in the fifth container.

### EXAMPLE 13

The experiment is conducted as in Example 12, but 0.01 g/l of 4-(2-pyridilazo)-resorcinol is introduced into the isopropyl alcohol.

After the third processing cycle, a red-brown (like the strong tea) coloring is apparent in the fifth container.

### EXAMPLE 14

The experiment is conducted as in the Example 2, but 35.2 g/l (8 g/l on the basis of zinc) of zinc sulfate is introduced into the fixing agent solution. 0.01 g/l of 2-carboxy-2′-hydroxy-5′-sulfoformazylbenzol is introduced into the waterless ethyl alcohol.

After the third processing cycle, a violet coloring is apparent in the fifth container.

Microscopic examination of the specimens processed in Examples 6-14 has shown that the specimens were processed with high quality.

As the examples show, the method of the invention permits more economical use of the processing liquid, since the coloring appearance is directly connected with the volume of liquid being transferred and the volume of specimens being processed. Moreover, the method of the invention permits monitoring of the status of the processing liquids. Renewal of the liquid is effected when loss in efficiency is detected.

### Industrial Usability

The method of the invention can be used in laboratories specializing in microscopic and biological research. The liquids used in such research are manufactured specially by the industry; the substances used in the method of the invention can be introduced into the liquids during manufacture thereof.

1. Method of processing histological and biological specimens by sequential immersions of specimens into processing liquids containing an aqueous solution of fixing agent, at least three dehydrating water containing liquids and at least three waterless dehydrating liquids, characterized in that a chemical marker is added to the water contained in the water solution of fixing agent, and an indicator, forming a colored composition with the chemical marker, is added to the waterless dehydrating liquid, and the waterless dehydrating liquid is renewed upon visual appearance of a coloring therein.

2. The method of claim 1, characterized in that a transition metal salt having an oxidation rate of 2+ in the amount of 2-40 g (on the basis of metal) per 1 liter of fixing agent water solution is used as a chemical marker.

3. The method of claim 1, characterized in that a salt of divalent iron is used as a chemical marker.

4. The method of claim 1, characterized in that a salt of divalent cobalt is used as a chemical marker.

5. The method of claim 1, characterized in that a salt of divalent nickel is used as a chemical marker.

6. The method of claim 1, characterized in that a salt of divalent copper is used as a chemical marker.

7. The method of claim 1, characterized in that a salt of zinc is used as a chemical marker.

8. The method of claim 1, characterized in that said indicator is a substance selected from the group consisting of 1,10-phenanthroline, diphenylthiocarbazone, 1-nitroso-2-naphthol, sodium diethyldithiocarbamate, 8-oxyquinoline, 1-(2-pyridilazo)-2-naphthol, 4-(2-pyridilazo)-resorcinol and 2-carboxy-2′-hydroxy-5′-sulfoformazylbenzol taken in the amount of 0.001-0.4 g per liter of waterless liquid.

9. The method of claim 1, characterized in that potassium or sodium thiocyanate taken in the amount of 5-10 g per liter of the waterless liquid is used as an indicator.

10. The method of claim 1, characterized in that an ethyl alcohol is used as a dehydrating liquid.

11. The method of claim 1, characterized in that an isopropyl alcohol is used as a dehydrating liquid.

12. The method of claim 1, characterized in that mixtures on the basis of ethyl and isopropyl alcohols are used as dehydrating liquids.
